(19) United States
(12) Patent Application Publication
(10) Pub. No.: US 2017/0253906 A1
(43) Pub. Date: Sep. 7, 2017

(54) MICROBIAL COLONY SPOTTING CHAMBER FOR CULTURE OF SINGLE CELL MICROBIAL COLONIES AND METHOD THEREOF

(71) Applicant: King Abdulaziz City for Science and Technology, Riyadh (SA)

(72) Inventor: Essam Jamil Alyamani, Riyadh (SA)

(21) Appl. No.: 15/062,142

(22) Filed: Mar. 6, 2016

(51) Int. Cl. C12Q 1/24; C12M 1/22; C12M 3/06; C12M 1/00; B01L 3/00

(52) U.S. Cl. CPC C12Q 1/24; C12M 23/34; B01L 3/502738; C12M 23/28; B01L 3/502715; C12M 23/16; C12M 23/10; B01L 3/502746; B01L 2200/141; B01L 2300/0864; B01L 2300/12; B01L 2400/082; B01L 2300/0877; B01L 2300/0609

(57) ABSTRACT

A disposable colony spotting chamber for generation of single cell microbial colonies is provided. The disposable colony spotting chamber includes an entry orifice through which clinical sample can be fed into the colony spotting chamber. The entry orifice opens into a network of micron sized microfluidics chambers which are interconnected by microfabricated channels. The microfluidics chambers and microfabricated channels ensure that the clinical sample is distributed equally amongst the microfluidics chambers. The disposable colony spotting chamber further includes a sample release button that is when pressed releases the clinical sample on a petri dish from microfluidics chambers.

### FIELD OF THE INVENTION

The present invention generally relates to a laboratory tool for microbiology work. More specifically, the present invention relates to an apparatus and method to produce pure single cell microbial colonies for further analysis.

### BACKGROUND OF THE INVENTION

Growth of single cell pure colonies of a microorganism on culture media is very important for further analysis of microorganisms, in research laboratories as well as pharmaceutical and clinical laboratories. Presently obtaining a single cell pure colony of microorganism on culture media is very tedious and requires a well trained skilled microbiology technician to perform this activity. In case of samples containing multiple microorganisms e.g. samples from urinary tract infections, it becomes even more difficult to obtain well separated pure single cell microbial colonies and requirement of trained and skilled microbiologist becomes indispensible.

Presently streak plate method or spread plate method is used to grow single cell pure microbial colonies. In these methods a diluted sample is streaked or spread on a petri-dish containing culture media with the help of a sterilized inoculation loop or spreader. The drawbacks being requirement of a well trained and skilled microbiology technician to perform these activities and increased chances of contamination and failure in obtaining well separated single cell colonies. This problem gets aggravated in cases of samples containing multiple microorganisms.

Trained and skilled microbiology technicians are not easily available and generally are in high demand; particularly in larger hospitals and clinical laboratories where a large number of samples are required to be cultured and analyzed daily. Chances of cross contamination of samples also increase if the same technician handles all or most of the samples.

Therefore, there exists a need for an improved method and apparatus to generate pure single cell microbial colonies that are well separated and easy to count without the use of cumbersome process and involvement of a highly skilled technician.

### SUMMARY OF THE INVENTION

The invention discloses a novel disposable colony spotting chamber for culturing of pure single cell microbial colonies in petri dish containing culture media, for further analysis and a method for obtaining pure single cell microbial colonies by using this colony spotting chamber. This invention reduces dependency on skilled microbiology technicians and enables easy handling of large number of samples in research laboratories, hospitals and clinical laboratories. This invention also helps in prevention of cross contamination of microbial cultures.

One embodiment of the invention discloses a method for generating pure single cell microbial colonies by using colony spotting chamber. The method includes steps of obtaining a clinical sample in a sterile sample container, extraction of microtitre of the clinical sample by micropipette and putting it in a sterile colony spotting chamber through sample filling orifice. The colony spotting chamber is then flipped, pressed and sample is released by pushing the sample release button, on a petri dish containing suitable culture media. The petri dish is then incubated at desired temperature for desired time period to allow microbial colonies to grow.

Another embodiment of the invention provides a disposable sterile colony spotting chamber for generating pure single cell microbial colonies. The colony spotting chamber includes multiple microfluidics chambers of equal sizes. These microfluidics chambers are interconnected to each other through microfabricated channels.

In a further embodiment of the invention colony spotting chamber includes a sample filling orifice through which the clinical sample containing the microorganism is fed to the colony spotting chamber. Clinical sample fed through sample filling orifice directly riches to microfabricated channels. The microfabricated channels are interconnect with microfluidics chambers and through these channels equal amount of sample reaches to each microfluidics chamber.

In another embodiment of invention each microfluidics chamber has a channel gate which remains closed to hold the sample in microfluidics chamber. Clinical sample is released from microfluidics chambers to petri dish by opening of these channel gates.

In a further embodiment of the invention the colony spotting chamber includes a sample release button which is connected internally to open the channel gates of the microfluidics chambers. The sample release button triggers complete release of the sample from the microfluidics chambers to a petri dish containing microbial growth media.

In another embodiment of the invention colony spotting chamber contains tow holding sides. These tow holding sides help in holding and manipulation of colony spotting chamber (100).

Additional features and advantages are realized through the techniques of the present invention. These embodiments and aspects of the invention are described in detail herein and are considered a part of the claimed invention. For a better understanding of the invention with advantages and features, refer to the description and to the drawings.

### BRIEF DECRIPTIONN OF THE DRAWINGS

The subject matter that is regarded as the invention is particularly pointed out and distinctly claimed in the claims at the conclusion of the specification. The foregoing and other aspects, features, and advantages of the invention are apparent from the following detailed description taken in conjunction with the accompanying drawings in which:

FIG. 1a, 1b, and 1c illustrates front, back and side views and various components of the disposable colony spotting chamber according to an embodiment of the invention.

FIG. 2 illustrates the schematic representation of method for obtaining culture of pure microbial single cell colonies from sample by using colony spotting chamber according to an embodiment of the invention; and

FIG. 3 illustrates a flow chart for a method according to an embodiment of the invention.

It is to be noted that the drawings presented are intended solely for the purpose of illustration and that they are, therefore, neither desired nor intended to limit the disclosure to any or all of the exact details of construction shown, except insofar as they may be deemed essential to the claimed invention.

### DETAILED DESCRIPTION

Several embodiments for a novel colony spotting chamber (100) are described. Although the present embodiments have been described with reference to specific example embodiments, it will be evident that various modifications and changes may be made to these embodiments without departing from the broader spirit and scope of the various embodiments.

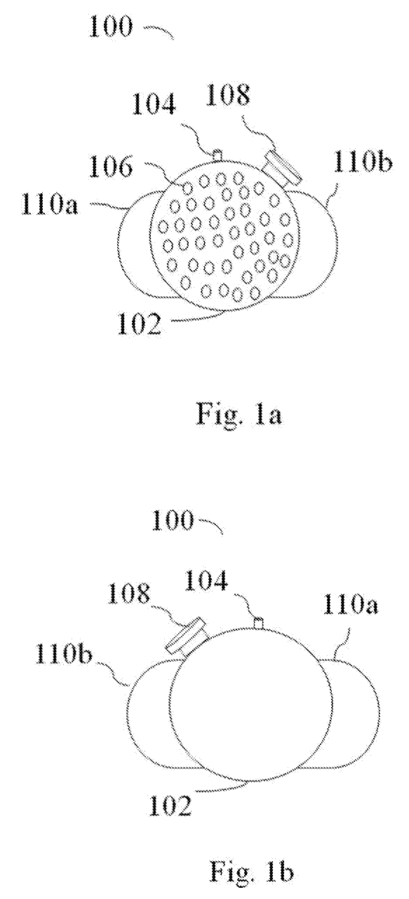

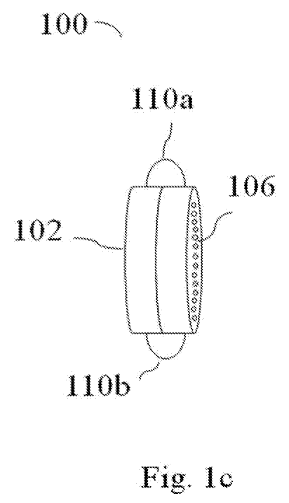

Referring to FIG. 1a that depicts a front view of a disposable sterile colony spotting chamber (100). In an embodiment of the invention the disposable sterile colony spotting chamber (100) is made up of disposable plastic material having transparent appearance, or an acrylic sheet. The disposable sterile colony spotting chamber (100) includes a base plate (102) that forms the main body part of the disposable sterile colony spotting chamber (100). The disposable sterile colony spotting chamber (100) further includes a sample filling orifice (104) through which a clinical sample solution containing microorganism for colonial generation could be dropped. The disposable sterile colony spotting chamber (100) further contains multiple sample containing chambers or microfluidics chambers (106) that are in fluid connection with the sample filing orifice (104). In an embodiment of the invention, the microfluidics chambers (106) are of equal volume so as to hold equal quantity of the clinical sample. In another embodiment of the invention, the multiple sample containing chambers or microfluidics chambers (106) are interconnected via microfabricated channels. In another embodiment of the invention each microfluidics chamber (106) has a channel gate, opening of which release the clinical sample on petri dish containing culture media. These channel gates remain closed till there opening is triggered.

Still referring to FIG. 1a, the disposable sterile colony spotting chamber (100) further includes a sample release button (108). The sample release button (108) helps in easy and complete discharge of the clinical sample from the sample containing chambers or microfluidics chambers (106) to a petri dish. The sample release button (108) is connected internally to open the channels gates of the microfluidics chambers (106) so as to completely release the clinical sample from the microfluidic chambers (106). Further, for easy holding and handling of the disposable sterile colony spotting chamber (100), there are provided two holding handles (110a and 110b) one on each side of the disposable sterile colony spotting chamber (100).

FIG. 1b, illustrates a back view of the sterile colony spotting chamber (100). Whereas FIG. 1c illustrates a side view of the sterile colony spotting chamber (100).

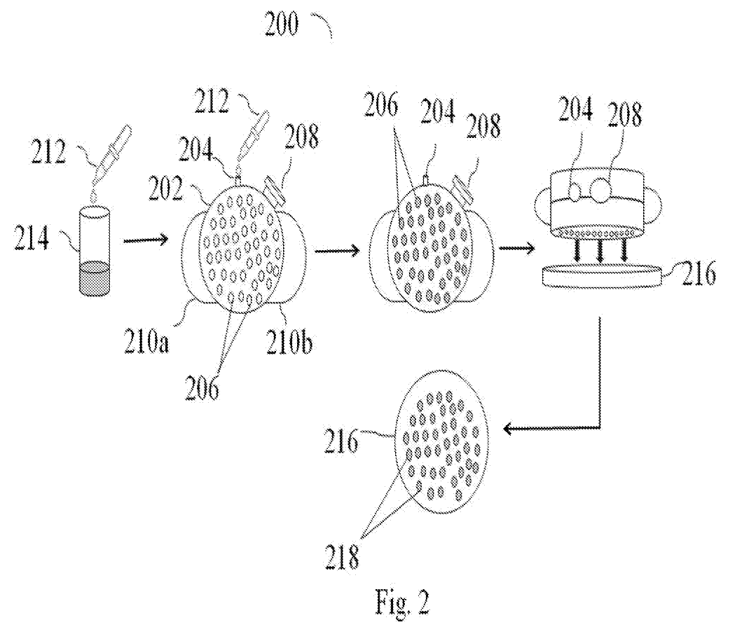

Now referring to FIG. 2 that illustrates a schematic representation of the method (200) for generating pure single cell microbial colonies from a clinical sample by using present colony spotting chamber (100). This depicts a sample container (214) that holds a clinical sample. The clinical sample contains microorganism of which a pure single cell colony is desired for further analysis. A microtitre of sample is extracted from the sample container (214) by a micropipette (212). Sample extracted by micropipette (212) is fed into a disposable colony spotting chamber (202) through an entry orifice (204). Sample fed through the entry orifice (204) reaches to microfluidics chambers (206) through microfabricated channels. Once clinical sample containing microorganisms are equally distributed in microfluidics chambers (206), the colony spotting chamber (202) is flipped on a petri dish (216) containing suitable culture media and sample release button (208) is pressed to open the channel gates and completely release the sample. The petri dish (216) is then incubated at suitable temperature for suitable time period to allow single cell microbial colonies (218) to grow.

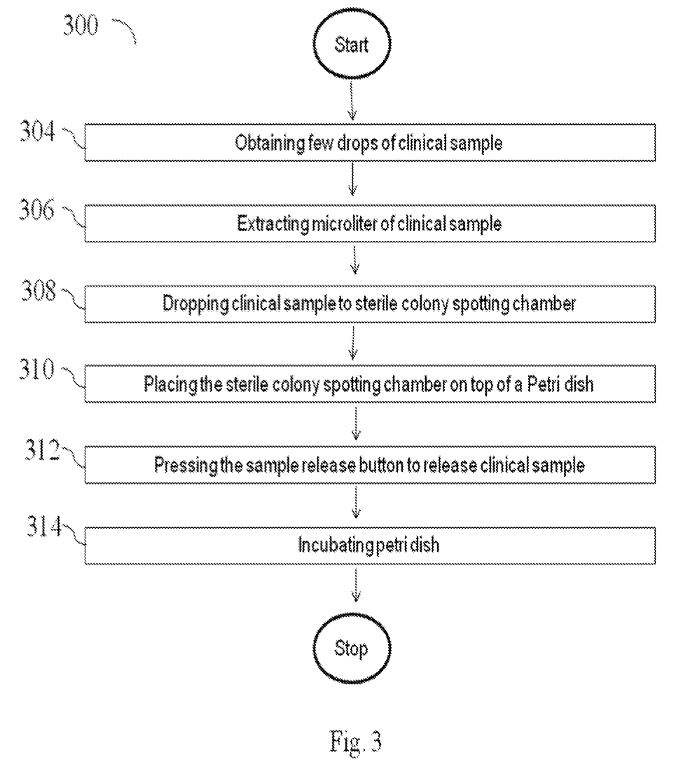

Now referring to FIG. 3 that illustrates a flow chart for method (300) of generating single cell microbial colonies from a clinical sample using colony spotting chamber of the present invention. References will be made to FIG. 2 in order to support various steps of the method 300.

The foregoing description and drawings comprise illustrative embodiments of the present invention. Having thus described exemplary embodiments, it should be noted by those ordinarily skilled in the art that the within disclosures are exemplary only, and that various other alternatives, adaptations, and modifications may be made within the scope of the present invention. Merely listing or numbering the steps of a method in a certain order does not constitute any limitation on the order of the steps of that method. Many modifications and other embodiments of the invention will come to mind to one ordinarily skilled in the art to which this invention pertains having the benefit of the teachings presented in the foregoing descriptions and the associated drawings. Although specific terms may be employed herein, they are used in a generic and descriptive sense only and not for purposes of limitation. Moreover, the present invention has been described in detail; it should be understood that various changes, substitutions and alterations can be made thereto without departing from the spirit and scope of the invention as defined by the appended claims. Accordingly, the present invention is not limited to the specific embodiments illustrated herein, but is limited only by the following claims

What is claimed is:

1. A method for generating single cell microbial colonies, the method comprising;
obtaining drops of a clinical sample containing the microorganism in a container;
extracting a microliter of the clinical sample;
feeding the clinical sample in a sterile colony spotting chamber;
flipping the sterile colony chamber on a petri dish containing a microbial culture media;
releasing the clinical sample on the petri dish; and
incubating the petri dish for growth of single cell microbial colonies.

2. The method of claim 1, wherein the sterile colony spotting chamber includes two holding handles.

3. The method of claim 1, wherein the sterile colony spotting chamber includes a plurality of microfluidics chambers.

4. The method of claim 3, wherein the plurality of microfluidics chambers are interconnected via microfabricated channels.

5. The method of claim 1, wherein the sterile colony spotting chamber includes a sample releasing button.

6. The method of claim 5, wherein the sample releasing button is configured to open the microfluidics chambers.

7. The method of claim 1, wherein the sterile colony spotting chamber is disposable and made up of, a disposable plastic, or an acrylic sheet.

8. The method of claim 1, wherein the sterile colony spotting chamber is of a spherical or oval shape.

9. A disposable sterile colony spotting chamber for growing single cell microbial colonies, comprising;
a plurality of microfluidics chambers;
a sample filling orifice configured to collect and distribute the sample equally to the microfluidics chambers; and
a sample release button configured to release the sample to a petri dish to obtain the single cell microbial colonies.

10. The disposable sterile colony spotting chamber of claim 9, wherein the plurality of microfluidics chambers are interconnected via microfabricated channels.

11. The disposable sterile colony spotting chamber of claim 9, wherein the microfluidics chambers have channel gates.

12. The disposable sterile colony spotting chamber of claim 11, wherein the opening of the channel gates releases the sample out of microfluidics chambers.

13. The disposable sterile colony spotting chamber of claim 9, wherein the sample release button is interconnected with channel gates.

14. The disposable sterile colony spotting chamber of claim 9, wherein the opening of channel gates are controlled by sample release button.

15. The disposable sterile colony spotting chamber of claim 9, wherein the plurality of microfluidics chambers are of equal volume.

16. The disposable sterile colony spotting chamber of claim 9, wherein the sterile colony spotting chamber is made up of a disposable plastic or an acrylic sheet.

17. The disposable sterile colony spotting chamber of claim 9, wherein the colony spotting chamber has tow holding sides.
